(19) United States
(12) Patent Application Publication
(10) Pub. No.: US 2024/0299706 A1
(43) Pub. Date: Sep. 12, 2024

(54) KEYED STEERABLE CATHETER DEVICES, SYSTEMS, AND METHODS

(71) Applicant: W. L. Gore & Associates, Inc., Newark, DE (US)

(72) Inventor: James R. Black, Flagstaff, AZ (US)

(21) Appl. No.: 18/573,696

(22) PCT Filed: Jun. 24, 2022
(86) PCT No.: PCT/US2022/034961

§ 371 Date: Dec. 22, 2023

Related U.S. Application Data
(60) Provisional application No. 63/214,592, filed on Jun. 24, 2021.

(51) Int. Cl. A61M 25/01; A61M 25/00

(52) U.S. Cl. CPC A61M 25/0147; A61M 25/005; A61M 2025/0004

(57) ABSTRACT

Disclosed herein are devices, systems, and methods relating to steerable catheters. A steerable catheter includes a first actuation line, an inner catheter, and an outer catheter. The inner catheter has an inner catheter proximal end, an inner catheter distal end, an inner catheter outer surface defining a keyway that is longitudinally-extending, and an inner catheter inner surface defining an inner catheter lumen. The outer catheter has an outer catheter proximal end, an outer catheter distal end, an outer catheter outer surface, and an outer catheter inner surface defining an outer catheter lumen and a spline that is longitudinally-extending and that projects radially inward into the outer catheter lumen. The outer catheter lumen is operable to receive the inner catheter with the spline of the outer catheter operable to key with the keyway of the inner catheter. The spline defines a spline lumen operable to receive the first actuation line.

### CROSS-REFERENCE TO RELATED APPLICATION

This application is a national phase application of PCT Application No. PCT/US2022/034961, internationally filed on Jun. 24, 2022, which claims the benefit of Provisional Application No. 63/214,592, filed Jun. 24, 2021, which are incorporated herein by reference in their entireties for all purposes.

### FIELD

The present disclosure relates generally to medical apparatuses, systems, and methods. More specifically, the disclosure relates to apparatuses, systems, and methods involving steerable catheters.

### BACKGROUND

Catheters are tubular medical devices that may be inserted into a body vessel, cavity, or duct and manipulated utilizing a portion that extends out of the body. Typically, catheters are relatively thin and flexible to facilitate advancement and retraction along non-linear paths. Catheters typically employ elongate flexible tubes made from synthetic plastic materials. Catheters may be employed for a wide variety of purposes, including the internal bodily positioning of diagnostic and/or therapeutic devices. For example, catheters may be employed to position internal imaging devices, deploy implantable devices (e.g., stents, stent grafts, vena cava filters), and/or deliver energy (e.g., ablation catheters). Desirable characteristics of catheter tubing include the ability to transfer forces from the proximal to the distal end of the catheter. It is also desirable for the catheter to be flexible so as to navigate tortuous paths within a body lumen without kinking. As well, it is also desirable for the catheter to be robust so as to withstand manipulation of a device within a body lumen during applied torques required for certain procedures.

Steerable mechanisms are widely available for catheter delivery and navigation in a variety of minimally invasive surgical procedures. They may be used to deliver or push a catheter or other device, such as a pacemaker lead, through tortuous anatomy or to locate the entrance to an artery or vein. Typical steering mechanisms involve the use of a pull wire, which is connected to the distal end of the catheter tip and actuatable at the proximal end of the catheter. This arrangement allows the tip of the catheter to proportionally bend in response to a tension force applied to the pull wire. By tensioning the pull wire, the catheter can assume various complex curves, depending on the respective lumen through which the pull wire passes. Oftentimes, these pull wires are positioned within a dedicated lumen, which increases manufacturing complexities (e.g., maintaining lumen tolerances along the length of the catheter). Manufacturing costs are often higher for this reason and at least because of the additional space required (e.g., in the handle and across the diameter of the catheter) to accommodate related components of the steering mechanism. As well, the tip of catheters using such pull wires may only be deflected in a single plane, which limits the effectiveness of this type of catheter in reaching many of the desired treatment sites.

Minimally invasive procedures benefiting from the use of steerable catheters include, among others, transcatheter mitral valve repair/replacement (TMVR) and transcatheter mitral chord repair/replacement (TMCR). The mitral valve controls blood flow between the chambers on the left side of the heart, which pumps oxygenated blood to the body. Two primary problems may occur with the mitral valve: narrowing (stenosis) or leakage (regurgitation). As a result, abnormal blood flow through the mitral valve causes the heart to work harder and will eventually lead to heart failure. TMVR and TMCR provide minimally invasive options to treating mitral valve stenosis, regurgitation, or some mixture of the two. These procedures typically involve multiple catheters accessing the mitral valve through complex navigation requiring movement in multiple planes. For example, to gain access to the mitral valve, an outer catheter may be tracked over an introducer from a puncture in the femoral vein, through the inferior vena cava and into the right atrium. The outer catheter may be punctured through a fossa in the interatrial septum and then advanced through the fossa and curved so that the distal end is directed over the mitral valve. Positioning of the distal end over the mitral valve may be accomplished by shape setting the outer catheter, such that it assumes the shape-set position when the introducer is retracted, and/or by steering of the outer catheter to the desired position using in-plane and off-plane deflections. These deflections can be facilitated by rotationally constraining nested catheters using a keyway and corresponding key, both of which should be sufficiently rigid to maintain the rotational relationship between the nested catheters. It may be appreciated that this approach serves merely as an example and other approaches, such as gaining access through the jugular vein, femoral artery, port access, or direct access, may be used.

To be cost effective and for ease of manufacturing, steerable catheters often involve use of polymer extrusions. As described above, steerable systems that include pull wires may require a separate lumen for each pull wire and employ keyways for rotational constraints. Polymer extrusions may be mass produced but have the drawback of manufacturing inconsistencies, especially when it comes to features extending along a measurable portion of the catheter such as a keyway or key profiled into a catheter. Variability in the polymer extrusion, and the keyway or key by extension, increases with increasing length of the polymer extrusion. This variability may lead to unnecessary compromises in the operation and overall reliability of the catheter.

### SUMMARY

The present disclosure provides improved apparatuses, systems, and methods involving catheters with multi-planar steering and keyed configurations. Among other things, embodiments of the present disclosure include nested catheters having an outer sheath with a keyed profile and a mating inner sheath. Such embodiments are useful for steerable and non-steerable sheaths and catheters and provide several advantages. For example, such embodiments employ a relatively simple construction that ensures more uniform structural features such as those that extend along a measurable length of a catheter (e.g., splines, keyways, etc.) as compared to that of more conventional methods. In addition, when used as a steerable catheter, the present disclosure may use components of the drive mechanism (e.g., nested actuation lines or pull wires positioned within the spline) to provide mechanical strength where it is desirable—e.g., between a spline that keys with a keyway—without requiring additional components. If desired, this mechanical strength may be further increased by adding additional components nested within the spline, the keyway, or both without departing from the scope of this disclosure. In another example, embodiments employing a single continuous catheter liner may ensure that air or fluid within an inner lumen of the catheter does not disadvantageously escape to other portions of the catheter having components that are not intended to come into contact with such fluid or air. In this way, degeneration and compromised operation of these components (such as the actuation lines) may be prevented.

According to one example (“Example 1”), a steerable catheter includes a first actuation line, an inner catheter, and an outer catheter. The inner catheter has an inner catheter proximal end, an inner catheter distal end, an inner catheter outer surface optionally defining a keyway that is longitudinally-extending, and an inner catheter inner surface defining an inner catheter lumen. The outer catheter has an outer catheter proximal end, an outer catheter distal end, an outer catheter outer surface, and an outer catheter inner surface optionally defining an outer catheter lumen and a spline. The spline is longitudinally extending and optionally projects radially inward into the outer catheter lumen. The outer catheter lumen is operable to receive the inner catheter with the spline of the outer catheter operable to key with the keyway of the inner catheter. The spline defines a spline lumen operable to receive the first actuation line.

According to another example further to Example 1 (“Example 2”), the steerable catheter further includes an outer catheter actuation ring coupled to the outer catheter distal end. The first actuation line is coupled to the outer catheter actuation ring.

According to another example further to Examples 1 or 2 (“Example 3”), the steerable catheter further includes a second actuation line where the inner catheter defines an inner catheter actuation line lumen from the inner catheter proximal end to the inner catheter distal end and operable to receive the second actuation line therethrough. The second actuation line extends from the inner catheter proximal end to the inner catheter distal end and coupled thereto.

According to another example further to Example 3 (“Example 4”), the steerable further includes an inner catheter actuation ring coupled to the inner catheter distal end. The second actuation line is coupled to the inner catheter actuation ring.

According to another example further to any one of Examples 1-4 (“Example 5”), each of the spline and the keyway is parallel with a central axis of the steerable catheter.

According to another example further to any one of Examples 1-5 (“Example 6”), the inner catheter includes at least one locking collar that defines the keyway of the inner catheter.

According to another example further to any one of Examples 1-6 (“Example 7”), the inner catheter includes a first elongated tubular member defining the inner catheter outer surface, and the keyway is defined by the first elongated tubular member.

According to another example further to any one of Examples 1-7 (“Example 8”), the outer catheter inner surface further defines a plurality of splines, and the inner catheter outer surface further defines a plurality of respective keyways.

According to another example further to Examples 1-8 (“Example 9”), the outer catheter inner surface defines two splines that are located about 180 degrees apart on an outer catheter circumference, and the inner catheter outer surface defines two respective keyways located 180 degrees apart on an inner catheter circumference.

According to another example further to any one of Examples 1-8 (“Example 10”), the outer catheter inner surface defines three splines that are located about 90 degrees apart on an outer catheter circumference, and the inner catheter outer surface defines three respective keyways located about 90 degrees apart on an inner catheter circumference.

According to another example further to any one of Examples 1-8 (“Example 11”), the outer catheter inner surface defines three splines that are located about 120 degrees apart on an outer catheter circumference, and the inner catheter outer surface defines three respective keyways located about 120 degrees apart on an inner catheter circumference.

According to another example further to any one of Examples 1-11 (“Example 12”), the steerable catheter includes a deflectable region at or around the outer catheter distal end, and the keyway extends along a length of the inner catheter toward the inner catheter distal end and terminate proximate the deflectable region of the outer catheter.

According to another example further to any one of Examples 1-12 (“Example 13”), the inner catheter is slidably received within the outer catheter along a length of the keyway.

According to another example further to any one of Examples 1-13 (“Example 14”), the steerable catheter includes a hemostatic seal between the outer catheter inner surface and the inner catheter outer surface.

According to another example (“Example 15”), a steerable catheter includes a first actuation line, an inner catheter, an outer catheter, and optionally a keyed locking collar. The first actuation line has a proximal portion and a distal portion. The inner catheter defines a first elongated tubular member having an inner catheter proximal end and an inner catheter distal end, and an inner catheter outer surface and an inner catheter inner surface defining an inner catheter lumen. The keyed locking collar optionally includes a keyway and is coupled to the inner catheter. The outer catheter defines a second elongated tubular member having an outer catheter proximal end and an outer catheter distal end, and an outer catheter outer surface and an outer catheter inner surface defining an outer catheter lumen, the outer catheter inner surface defining a spline. The spline is optionally longitudinally extending and optionally projects radially inward into the outer catheter lumen. The outer catheter lumen is operable to receive the inner catheter therethrough with the spline keyed with the keyway. The spline defines a spline lumen slidably receiving the first actuation line. The first actuation line optionally extends along the outer catheter, and the distal portion of the first actuation line is optionally coupled proximate the distal end of the outer catheter.

According to another example further to Example 15 (“Example 16”), the keyed locking collar is integrally formed with the first elongated tubular member of the inner catheter.

According to another example further to Examples 15 or 16 (“Example 17”), the keyed locking collar radially extends beyond an outer diameter of the inner catheter.

According to another example further to any one of Examples 15-17 (“Example 18”), a length of the keyed locking collar is less than a length of the inner catheter.

According to another example further to any one of Examples 15-18 (“Example 19”), the outer catheter inner surface defines two splines that are located about 180 degrees apart on an outer catheter circumference, and the inner catheter outer surface defines two respective keyways located about 180 degrees apart on an inner catheter circumference.

According to another example further to any one of Examples 15-19 (“Example 20”), the outer catheter inner surface defines three splines that are located about 90 degrees apart on the outer catheter circumference, and the inner catheter outer surface defines three respective keyways located about 90 degrees apart on the inner catheter circumference.

According to another example (“Example 21”), a method of treating a patient. The method optionally includes obtaining a steerable catheter that is similar to those disclosed elsewhere herein, including those relating to any one of Examples 1-20. The method includes advancing the outer catheter into a body lumen. The method includes advancing the inner catheter into the outer catheter thereby engaging the spline with the keyway. The method optionally includes actuating the first actuation line to thereby cause movement at the distal end of the outer catheter.

According to another example further to Example 21 (“Example 22”), the steerable catheter further comprises a handle at the outer catheter proximal end. The handle is operatively coupled to the first actuation line such that actuation of the handle causes actuation of the first actuation line. Actuating the first actuation line optionally includes performing at least one actuation of the handle.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a perspective view of a catheter system in accordance with an embodiment;

FIG. 2A is a side elevation view of an outer catheter for the catheter system in accordance with an embodiment;

FIG. 2B is a side elevation view of a keyed mandrel for forming the catheter system in accordance with an embodiment;

FIG. 2C is a side elevation view of section C-C in FIG. 2A in accordance with an embodiment;

FIG. 2D is a side elevation view of section A-A in FIG. 2A in accordance with an embodiment;

FIG. 2E is a side elevation view of an inner catheter for the catheter system in accordance with an embodiment;

FIG. 3 is a side elevation view of an inner catheter with a keyed locking collar for the catheter system in accordance with an embodiment; and

FIG. 4 is a flowchart of a method for treating a patient in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

“Steerable” is defined as the ability to direct the orientation of a portion of a catheter distal to a steerable segment at an angle with respect to a portion of a catheter proximal to the steerable segment. “Steering” may include any known method of steering that may be utilized to direct the orientation of the portion of the catheter distal to the steerable segment at an angle with respect to the portion of the catheter proximal to the steerable segment, including methods that utilize more than one steerable segment. Such methods may include, without limitation, use of remote application of force (e.g., electrical (e.g., wired or wireless), mechanical, hydraulic, pneumatic, magnetic, etc.) with transmission of that force by various means including pull and/or push wires, hydraulic lines, air lines, magnetic coupling, or electrical conductors including without limitation transmission by manipulation of push and/or pull wires, filaments, tubes, and/or cables. In addition, the catheter body may be constructed to have segments with differing flexibility or compression properties from the other segments of the catheter body. In an embodiment having an inner tubular body and an outer tubular body, the outer tubular body may have one or more steerable segments with push/pull wires anchored to the distal end of the steerable segments and extending through one or more lumens of the outer tubular wall to attachment to the steering control in the handle. Steering of the outer tubular body may steer the inner tubular body as well. In a variation, the inner tubular body may be steerable and steering of the inner tubular body may steer the outer tubular body as well.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

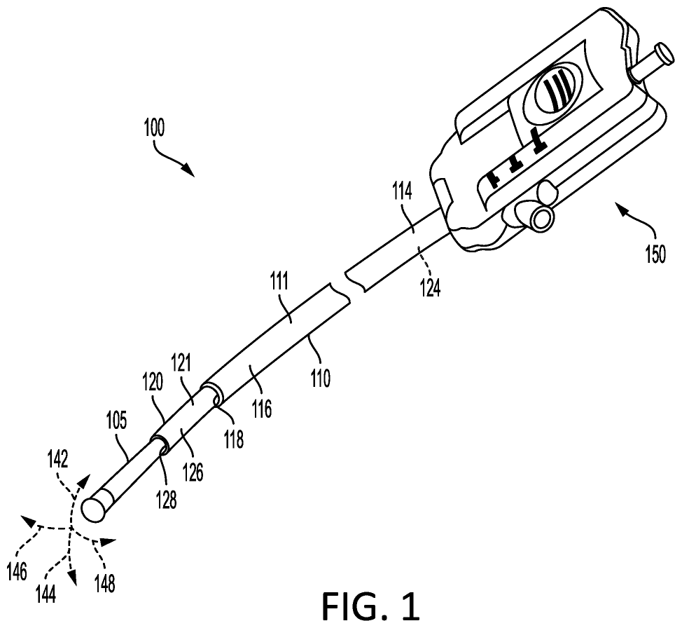

FIG. 1 shows a catheter system 100 in accordance with an embodiment of the present disclosure. With reference to this figure, an embodiment of a catheter system 100 includes multiple catheters. The catheter system 100 comprises an outer catheter 110, having an outer catheter proximal end 114, an outer catheter distal end 116, and an outer catheter lumen 118 therethrough, and an inner catheter 120, having an inner catheter proximal end 124, an inner catheter distal end 126, and inner catheter lumen 128 therethrough. As shown, the inner catheter 120 is positioned coaxially within the outer catheter lumen 118. The outer catheter distal end 116, the inner catheter distal end 126, or both are sized to be passable to a body cavity, typically through a body lumen such as a vascular lumen. The outer catheter lumen 118 is sized for the passage of the inner catheter 120. The inner catheter lumen 128 is sized for the passage of a variety of devices therethrough.

The outer catheter 110, the inner catheter 120, or both have steering mechanisms, embodiments of which will be described later in detail, to position the outer catheter distal end 116, the inner catheter distal end 126, or both in desired directions. The outer catheter 110 and the inner catheter 120 may be independently steerable (e.g., such that the outer catheter 110 may be steerable in a first direction and the inner catheter 120 may be steerable in a second direction that is different form the first direction). Together, the outer catheter 110 and the inner catheter 120 may form compound curvatures in a single direction. Tools may then be advanced through the outer and inner catheters 110, 120, through the compound curve, toward a desired direction that leads toward its target. Steering of the outer catheter 110 and inner catheter 120 may be achieved by actuation of one or more steering mechanisms. Actuation of the steering mechanisms is achieved with the use of actuators which are typically located on handle 150 connected with each of the outer and inner catheters 110, 120.

In embodiments, catheters may be shape set in addition to being steerable. Shape setting involves setting a specific curvature (e.g., via heat or via a shape-memory alloy) in the catheter prior to usage. Because catheters are generally flexible, loading of the catheter on a guidewire or other introducer straightens the shape-set catheter as the introducer progresses through the shaped region. After being positioned in a desired location within the patient, the introducer can be removed, and the catheter is allowed to return to its shape set. As described previously, curvatures may be formed in the outer and inner catheters 110, 120, for example, by shape setting or steering. To provide a higher degree of control and variety of possible curvatures, steering mechanisms may be used to create the curvatures and position the catheters. In some embodiments, the steering mechanisms comprise actuation lines (e.g., cables or pull wires) within the wall of the catheter.

As described in further detail hereinafter, the outer catheter 110 may include an actuation line (e.g., a cable or pull wire, not shown) that is slidably received within lumens extending longitudinally within the wall of the outer catheter 110. In embodiments, an entrance to the lumens within the wall of the catheter may be coincident with the outer catheter outer surface 111. By applying tension generally in the proximal direction, the actuation line may cause the outer catheter distal end 116 to curve in the direction of a first actuation line (not shown) as illustrated by arrow 142. Having a second actuation line (not shown) on an opposite side of the catheter will similarly cause the outer catheter distal end 116 to curve in the opposite direction, as illustrated by arrow 144, when tension is applied to that actuation line. This arrangement allows for the distal end to be steered in opposite directions, which allows for correcting or adjusting a curvature during operation. For example, if tension is applied to one actuation line to create a curvature, the curvature may be lessened by some combination of lessening the tension on the corresponding actuation line and applying tension to the diametrically opposite actuation line. There is a combination of actuation lines (e.g., four pull wires evenly spaced about outer catheter 110) that allows curvature of the distal end in at least four directions illustrated by arrows 142, 144, 146, 148. It may be appreciated that these arrows also pertain to the inner catheter 120. For example, actuation lines in the inner catheter 120 may create a second curve of the inner catheter 120 and the actuation lines.

Such actuation lines and associated lumens may be placed in any arrangement (e.g., in singles or in pairs, symmetrically or asymmetrically, etc.), and any number of actuation lines (e.g., one, two, three, four, etc.) may be used. Such arrangements may allow curvature in any direction and about various axes and planes. The actuation lines may be fixed at any location along the length of the catheter by any suitable method, such as gluing, tying, soldering, potting, etc. When tension is applied to the actuation line, curvature forms from the point of attachment of the actuation line toward the proximal direction. Therefore, curvatures may be formed throughout the length of the catheter depending on the locations of the points of attachment of the actuation lines. In an arrangement, the actuation lines will be attached near the distal end of the catheter, optionally to an actuation ring embedded in the outer catheter 110 as discussed further hereinafter. In some such embodiments, a second set of actuation lines may be coupled to another actuation ring embedded at a different location in the catheter. In addition, depending on the application, the lumens that house the actuation lines may be straight or may be curved.

The illustrated arrows correspond to articulated positions of the catheter system 100 that may be useful in accessing the mitral valve of a patient. For example, in a method of using the catheter system 100 for accessing the mitral valve, to gain access to the mitral valve, the outer catheter 110 may be tracked over an introducer (e.g., a dilator or guidewire) from a puncture in the patient's vasculature into the patient's heart (e.g., into the femoral vein and through the inferior vena cava and into the right atrium). The outer catheter 110 may then be punctured through a fossa in the interatrial septum and then advanced through the fossa and curved by a first curve so that the distal end is directed over the mitral valve. Positioning of the outer catheter distal end 116 over the mitral valve may be accomplished by shape setting of the outer catheter 110, wherein the outer catheter 110 assumes this position when the dilator and guidewire are retracted, and/or by steering of the outer catheter 110 to the desired position. It may be appreciated that this approach serves merely as an example and other approaches, such as gaining access through the jugular vein, femoral artery, port access, or direct access, may be used.

In embodiments, the steerable catheter may include a deflectable member 105 at or around the inner catheter distal end 126, and the keyway may extend along a length of the inner catheter 120 toward the inner catheter distal end 126 and terminate proximate the deflectable member 105 of the outer catheter 110. FIG. 1 shows an embodiment of a catheter system 100 that includes such a deflectable member 105. As noted prior, the catheter may be flexible and capable of bending to follow the contours of a body vessel into which it is being inserted. The deflectable member 105 may be disposed at an inner catheter distal end 126 of the catheter system 100. The catheter system 100 includes a handle 150 that may be disposed at the outer catheter proximal end 114, the inner catheter proximal end 124, or both. During a procedure where the deflectable member 105 is inserted into the body of a patient, the handle 150 and a portion of the catheter system 100 remain outside of the body. The user (e.g., physician, technician, interventionalist) of the catheter system 100 may control the position and various functions of the catheter system 100. For example, the user may hold the handle 150 and manipulate a portion thereof to control a deflection of the deflectable member 105. In this regard, the deflectable member 105 may be selectively deflectable. The handle 150 and the manipulatable portion thereof may be configured such that the manipulatable portion of the handle 150 relative to the handle 150 may be maintained, thereby maintaining the selected deflection of the deflectable member 105. Such maintenance of position may at least partially be achieved by, for example, friction (e.g., friction between a slide and a stationary portion of the handle 150), detents, and/or any other appropriate means. The catheter system 100 may be removed from the body by pulling (e.g., pulling the handle 150). Furthermore, the user may insert an interventional device (e.g., a diagnostic device and/or therapeutic device) through an interventional device inlet. The user may then feed the interventional device through the catheter system 100 to move the interventional device to the inner catheter distal end 126 of the catheter system 100. For example, electrical interconnections between an image processor and the deflectable member may be routed through an electronics port and through the catheter system 100.

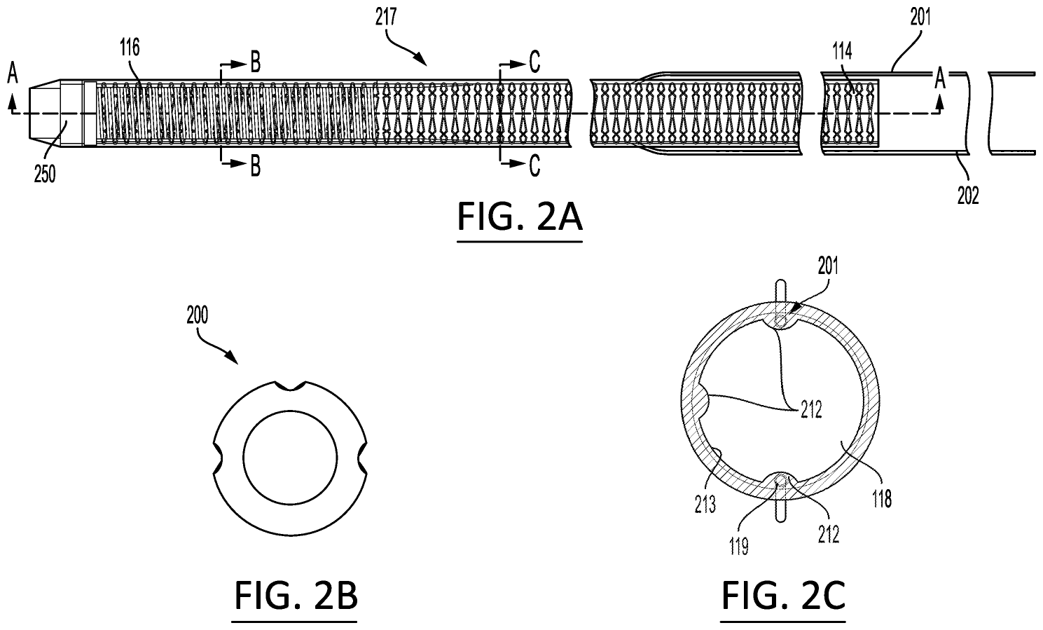

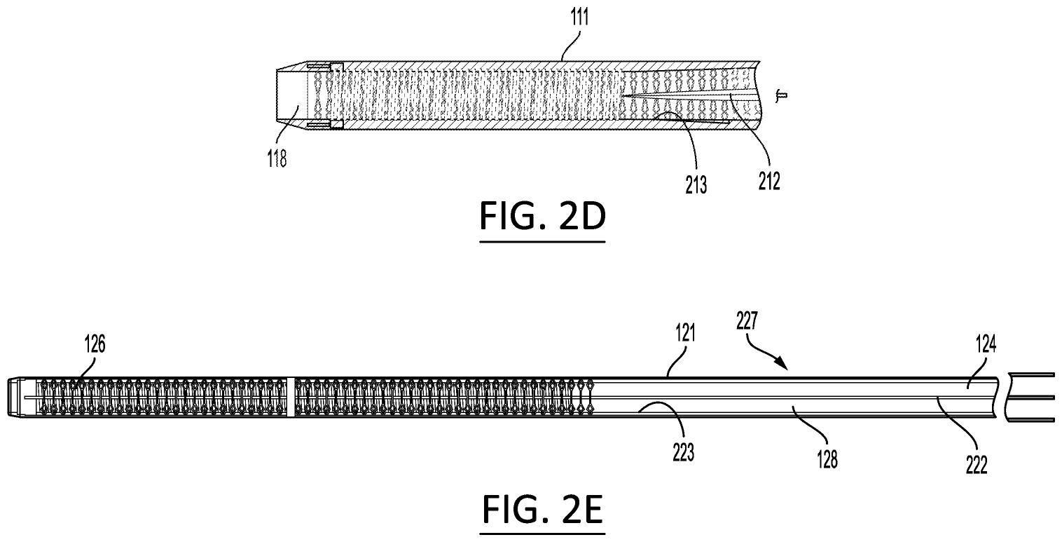

FIGS. 2A-2E show various views of outer and inner catheters 110, 120 in the catheter system 100 in accordance to a first embodiment of the present disclosure. FIG. 2A shows a side elevation view of an outer catheter 110 for the catheter system 100. FIG. 2B shows a side elevation view of a keyed mandrel 200 for forming the catheter system 100. FIG. 2C shows a side elevation view of section C-C in FIG. 2A. FIG. 2D shows a side elevation view of section A-A in FIG. 2A. And FIG. 2E shows a side elevation view of an inner catheter 120 for the catheter system 100. As is a theme throughout this disclosure, in embodiments, one or more features discussed in relation to one catheter (e.g., the outer catheter) may be included in another catheter (e.g., the inner catheter).

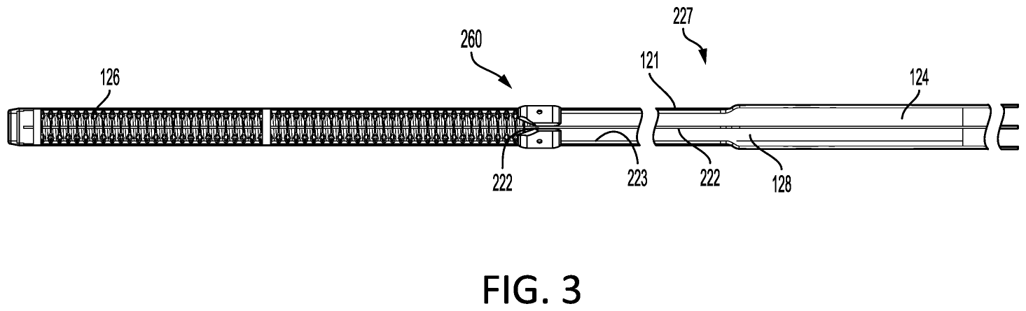

The system shown in FIGS. 2A-2E is provided as an example of the various features of the system and, although the combination of those illustrated features is clearly within the scope of invention, that example and its illustration is not meant to suggest the inventive concepts provided herein are limited from fewer features, additional features, or alternative features to one or more of those features shown in FIGS. 2A-2E. For example, in various embodiments, the components and/or characteristics of the system shown in FIGS. 2A-2E may include the other components and/or characteristics described with reference to FIG. 1 or FIG. 3. It should also be understood that the reverse is true as well. One or more of the components depicted in FIGS. 2A-2E can be employed in addition to, or as an alternative to components depicted in FIG. 1 or FIG. 3. For example, the components and/or characteristics of the system shown in FIGS. 2A-2E may be employed in connection with the components and/or characteristics of the other device shown in FIG. 3.

In the first embodiment of the present disclosure, a steerable catheter may include a first actuation line 201, an inner catheter 120, and an outer catheter 110. The steerable catheter can be similar to those catheter systems disclosed elsewhere herein, including the catheter system 100 as is shown here. The first actuation line 201, for example, may be a cable or pull wire. The inner catheter 120 may have an inner catheter proximal end 124, an inner catheter distal end 126, an inner catheter outer surface 121 defining a keyway 222 that is longitudinally extending, and an inner catheter inner surface 223 defining an inner catheter lumen 128. The outer catheter 110 may have an outer catheter proximal end 114, an outer catheter distal end 116, an outer catheter outer surface 111, and an outer catheter inner surface 213 defining an outer catheter lumen 118 and a spline 212. The spline 212 may be longitudinally extending and may project radially inward into the outer catheter lumen 118. The outer catheter lumen 118 may be operable to receive the inner catheter 120 with the spline 212 of the outer catheter 110 operable to key with the keyway 222 of the inner catheter 120.

The spline 212 may define a spline lumen 119 operable to receive (e.g., to slidably receive) the first actuation line 201. Having the having the first actuation line 201 located within the spline 212 may provide additional mechanical support to resist abrasion and torque. Additional relatively high durometer material monofilaments, or mandrels may be placed in this spline 212 and positioned about the first actuation line 201 to make the spline 212 more robust (e.g., increasing its mechanical strength). Additionally, braid or coil reinforced liners can be used in the spline 212 (e.g., to create the spline lumen 119) for additional mechanical support. It should be noted that in addition to the first actuation line 201, the spline lumen 119 may provide various other functions as a working lumen such as the ability to transfer fluid (e.g., contrast media or a diluent), the ability to receive electrical wires or leads (e.g., for tool operation, pressure measurements, etc.), and the like, in any combination, without departing from the scope of this disclosure. As well, the present disclosure may include one or more such working lumens apart from the spline lumens 119 that receive either the first actuation line 201 or any other actuation line without departing from the scope of this disclosure.

Relative motion between the inner catheter 120 and the outer catheter 110 may be such that the two can freely slide longitudinally but are constrained rotationally (e.g., by the combination of the spline 212 and keyway 222). The splines 212 may contribute to these constraints. For example, at or around a point along the length of the outer catheter 110, the outer catheter inner surface 213 may transition from a profiled inside diameter (e.g., one with splines 212) to a smooth diameter. Such embodiments include the spline 212 extending from at or around the outer catheter proximal end 114 toward the outer catheter distal end 116 and terminating short of the outer catheter distal end 116. This transition can happen at any length along the outer catheter 110. In embodiments, the transition occurs at the transition into the deflectable member 105 (discussed further above) of the catheter. Transitioning the outer catheter inner surface 213 may advantageously provide a mechanical hard stop for the inner catheter 120 because the keyway 222 is no longer guided by the spline 212. In addition, or in alternative, transitioning the outer catheter inner surface 213 may advantageously provide a geometry at the distal face that can be tipped (e.g., via traditional catheter tipping techniques).

In embodiments, the inner catheter 120 may be slidably received within the outer catheter 110 along a length of the keyway 222. In embodiments, each of the spline 212 and the keyway 222 may be parallel with a central axis of the steerable catheter. In embodiments, the inner catheter 120 may include a first elongate tubular member 227 defining the inner catheter outer surface 121. In such embodiments, the keyway 222 may be defined by the first elongate tubular member 227. For example, the length of the keyway 222 may extend from the inner catheter proximal end 124 to the inner catheter distal end 126. In other examples, the length of the keyway 222 may extend from the inner catheter proximal end 124 toward the inner catheter distal end 126 and terminate short of the inner catheter distal end 126 (e.g., before a coiled or braided portion of the inner catheter 120.) In alternative, the keyway 222 may extend from the inner catheter distal end 126 toward the inner catheter proximal end 124 and terminate short of the inner catheter proximal end 124. In some embodiments, the keyway 222 may intermediately extend along a length of the inner catheter 120 such that the keyway 222 terminates short of both the inner catheter proximal end 124 and the inner catheter distal end 126. In any case, the length of the keyway 222 (and other dimensions such as the depth) may vary between these examples so long as the inner catheter 120 remains slidably received within the outer catheter 110 (e.g., at the spline 212) along a length of the keyway 222.

Spacing multiple splines 212 apart about the outer catheter inner surface 213 may provide a balanced fit and ensure orientation of the inner catheter 120 within the outer catheter 110. In that regard, the outer catheter inner surface 213 may define a plurality of splines 212, and the inner catheter outer surface 121 may define a plurality of respective keyways 222. And any number (e.g., one, two, three, four, etc.) of splines 212 or keyways 222 may be used. In embodiments, the outer catheter inner surface 213 may define two splines 212 that are located about 180 degrees apart on an outer catheter circumference, and the inner catheter outer surface 121 may define two respective keyways 222 located 180 degrees apart on an inner catheter circumference. While providing a balanced fit, two such splines 212 alone may allow for the inner catheter 120 to fit within the outer catheter 110 under two orientations (e.g., a first orientation at 0 degrees rotation about the central axis and a second orientation at 180 degrees rotation about the central axis). As such, in some embodiments, three or more splines 212 may be used so as to only allow one such orientation. For example, in embodiments, the outer catheter inner surface 213 may define three splines 212 that are located about 90 degrees apart on an outer catheter circumference, and the inner catheter outer surface 121 may define three respective keyways 222 located about 90 degrees apart on an inner catheter circumference. In embodiments, the outer catheter inner surface 213 may define three splines 212 that are located about 120 degrees apart on an outer catheter circumference, and the inner catheter outer surface 121 may define three respective keyways 222 located about 120 degrees apart on an inner catheter circumference. Although discussed in terms of having a respective number of splines 212 and keyways 222, it should be noted that the number of splines 212 and keyways 222 may differ in embodiments without departing from the scope of this disclosure.

In embodiments, the steerable catheter may comprise an outer catheter actuation ring 250 coupled to the outer catheter distal end 116. The first actuation line 201 may be coupled to the outer catheter actuation ring 250. In embodiments, the steerable catheter may include a second actuation line 202. For example, the inner catheter 120 may define an inner catheter 120 actuation line lumen from the inner catheter proximal end 124 to the inner catheter distal end 126 and operable to receive the second actuation line 202 therethrough. The second actuation line 202 may extend from the inner catheter proximal end 124 to the inner catheter distal end 126 and may be coupled thereto. In embodiments, the steerable catheter may include an inner catheter 120 actuation ring coupled to the inner catheter distal end 126. And the second actuation line 202 may be coupled to the inner catheter 120 actuation ring. Each actuation ring may be embedded into a corresponding catheter (e.g., the outer catheter 110 or the inner catheter 120).

As noted below, in embodiments, a catheter liner 215 may be a single continuous catheter liner 215 extending from the outer catheter proximal end 114 to the outer catheter distal end 116. The catheter liner 215 may define the outer catheter inner surface 213. Such a catheter liner 215 may advantageously be devoid of seams or joints required to connect separate portions of non-continuous liners. These seams or joints often create points of failure for a catheter assembly because air or fluid may escape thereat, finding their way into other portions of the catheter assembly. This event disadvantageously affects components of the catheter assembly intended to be kept away from air or fluid escaping from the catheter liner 215, such as the first actuation line 201, and can lead to corrosion or compromised operation thereof.

FIG. 3 shows a side elevation view of an inner catheter 120 with a keyed locking collar 260 for the catheter system in accordance with a second embodiment of the present disclosure.

The device shown in FIG. 3 is provided as an example of the various features of the device and, although the combination of those illustrated features is clearly within the scope of invention, that example and its illustration is not meant to suggest the inventive concepts provided herein are limited from fewer features, additional features, or alternative features to one or more of those features shown in FIG. 3. For example, in various embodiments, the components and/or characteristics of the device shown in FIG. 3 may include the other components and/or characteristics described with reference to FIG. 1 or FIGS. 2A-2E. It should also be understood that the reverse is true as well. One or more of the components depicted in FIG. 3 can be employed in addition to, or as an alternative to components depicted in FIG. 1 or FIGS. 2A-2E. For example, the components and/or characteristics of the device shown in FIG. 3 may be employed in connection with the other components and/or characteristics of the other device shown in FIGS. 2A-2E.

In a second example of the present disclosure, the inner catheter 120 of a steerable catheter may include at least one keyed locking collar 260 that defines the keyway 222 of the inner catheter 120. In all other respects, the steerable catheter may be similar to those described elsewhere herein, including that of the first embodiment. For example, the steerable catheter may include a first actuation line 201, an inner catheter 120, a keyed locking collar 260, and an outer catheter 110. The first actuation line 201 may have a proximal portion and a distal portion. The inner catheter 120 may define a first elongated tubular member 227 having an inner catheter proximal end 124 and an inner catheter distal end 126, and an inner catheter outer surface 121 and an inner catheter inner surface defining an inner catheter lumen 128. The keyed locking collar 260 may include a keyway 222 (similar to other keyways described elsewhere herein) and may be coupled to the inner catheter 120. The outer catheter 110 may define a second elongated tubular member 217 having an outer catheter proximal end and an outer catheter distal end, and an outer catheter outer surface 111 and an outer catheter inner surface defining an outer catheter lumen, the outer catheter inner surface defining a spline. The spline may be longitudinally extending and may project radially inward into the outer catheter lumen. The outer catheter lumen may be operable to receive the inner catheter 120 therethrough with the spline keyed with the keyway 222. The spline may define a spline lumen 119 slidably receiving the first actuation line 201. The first actuation line 201 may extend along the outer catheter 110, and the distal portion of the first actuation line 201 may be coupled proximate the distal end of the outer catheter 110.

The keyed locking collar 260 may be coupled to the inner catheter 120 as a separate component thereto or a profiled feature thereof. In embodiments, the keyed locking collar 260 may be a separate component that is fixedly or removably coupled to the inner catheter 120. When the keyed locking collar 260 is removably coupled, its position relative to the inner catheter 120 may be adjustable. In embodiments, the keyed locking collar 260 may be integrally formed with the first elongated tubular member of the inner catheter 120 such that the keyed locking collar 260 is a profiled feature of the inner catheter 120. In this example, integrally formed means integrally manufactured therewith. For instance, the keyed locking collar 260 may be integrally formed by overmolding, insert molding, skiving, split-die, or some other similar manufacturing processes. Regardless of whether the keyed locking collar 260 is a separate component or a profiled feature of the inner catheter 120, the keyed locking collar 260 may radially extend beyond an outer diameter of the inner catheter 120.

Similar to the keyway 222 discussed above, in embodiments, a length of the keyway 222 and, by extension, the keyed locking collar 260 may vary between embodiments. For example, as noted, the length of the keyway 222 may extend from the inner catheter proximal end 124 to the inner catheter distal end 126. In other examples, the length of the keyway 222 may extend from the inner catheter proximal end 124 toward the inner catheter distal end 126 and terminate short of the inner catheter distal end 126. In alternative, the keyway 222 may extend from the inner catheter distal end 126 toward the inner catheter proximal end 124 and terminate short of the inner catheter proximal end 124. In some embodiments, the keyway 222 may intermediately extend along a length of the inner catheter 120 such that the keyway 222 terminates short of both the inner catheter proximal end 124 and the inner catheter distal end 126. In any case, the length of the keyway 222 (and other dimensions such as the depth) may vary between these examples so long as the inner catheter 120 remains slidably received within the outer catheter 110 (e.g., at the spline) along a length of the keyway 222. As well, the length of the keyed locking collar 260 may similarly vary in proportion to the keyway 222, the inner catheter 120, or both. For example, in embodiments, the length of the keyed locking collar 260 may be less than or equal to a length of the inner catheter 120, the length of the keyway 222 may be less or equal to the length of the keyed locking collar 260, or both.

As was the case in the first embodiment, spacing multiple splines apart about the outer catheter inner surface may provide a balanced fit and ensure orientation of the inner catheter 120 within the outer catheter. In embodiments, the outer catheter inner surface may define two splines that are located about 180 degrees apart on an outer catheter circumference, and the inner catheter outer surface 121 may define two respective keyways 222 located about 180 degrees apart on an inner catheter circumference. In embodiments, the outer catheter inner surface may define three splines that are located about 90 degrees apart on the outer catheter circumference, and the inner catheter outer surface 121 may define three respective keyways 222 located about 90 degrees apart on the inner catheter circumference. In embodiments, the outer catheter inner surface may define three splines that are located about 120 degrees apart on an outer catheter circumference, and the inner catheter outer surface 121 may define three respective keyways 222 located about 120 degrees apart on an inner catheter circumference. As noted above, although discussed in terms of having a respective number of splines and keyways 222, it should be noted that the number of splines and keyways 222 may differ in embodiments without departing from the scope of this disclosure.

In embodiments, the steerable catheter may include a hemostatic seal between the outer catheter inner surface and the inner catheter outer surface 121. The hemostatic seal may include a silicone outer tube and an inner film tube and may be pressured to create the hemostatic seal. Such a seal may easily adapt to the profiles of devices inserted into the outer catheter while minimizing blood loss and accommodating multiple wires and catheters simultaneously, for example.

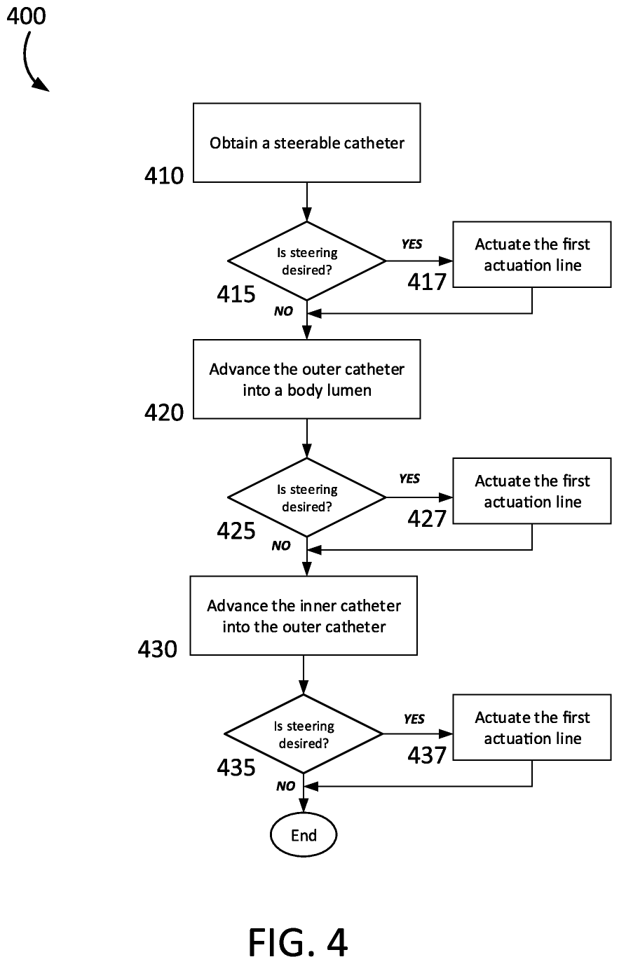

The present disclosure also includes a method 400 of treating a patient as shown in FIG. 4. At step 410, the method 400 may include obtaining a steerable catheter that is similar to those disclosed elsewhere herein, including the catheter system 100. At step 415, it may be determined whether it is desirable to steer the catheter. If so, the method 400 may continue by actuating the first actuation line at step 417 before proceeding to step 420, but if not, the method 400 may continue from step 415 to step 420. At step 420, the method 400 may include advancing the outer catheter into a body lumen. At step 425, it may be determined whether it is desirable to steer the catheter. If so, the method 400 may continue by actuating the first actuation line at step 427 before proceeding to step 430, but if not, the method 400 may continue from step 425 to step 430. At step 430, the method 400 may include advancing the inner catheter into the outer catheter thereby engaging the spline with the keyway. At step 435, it may be determined whether it is desirable to steer the catheter. If so, the method 400 may continue by actuating the first actuation line at step 437 before ending, but if not, the method 400 may end after step 435.

As alluded to above, it is apparent that the method 400 may include actuating the first actuation line (e.g., at steps 417, 427, and 437) at any point during the method 400 as desired. Such actuations may involve manipulation of a portion of the catheter assembly. For example, in embodiments, the method 400 may include actuating the first actuation line to thereby cause movement at the outer catheter distal end so as to navigate the catheter through a patient's body lumens. As noted prior, these actuations may involve simple movement or complex movements (e.g., movements about various axes and in multiple planes). In embodiments, the steerable catheter further comprises a handle at the outer catheter proximal end. The handle may be operatively coupled to the first actuation line such that actuation of the handle causes actuation of the first actuation line. In some such embodiments, actuating the first actuation line includes performing at least one actuation of the handle.

The present disclosure also includes a method of manufacturing a catheter. The method can include placing a catheter liner (see, e.g., catheter liner 215 in FIG. 2C) around a keyed mandrel (see, e.g., keyed mandrel 200 in FIG. 2C). In embodiments, the catheter liner may be a single continuous catheter liner extending from the proximal end of the catheter to the distal end of the catheter. The method can include placing over the catheter liner and into at least one keyway of the keyed mandrel an actuation line assembly comprising an actuation line liner and an actuation line therein. The method can include braiding a braid (see, e.g., braid 291 in FIG. 2C) over the catheter liner and the actuation line thereby securing the actuation line assembly into the at least one keyway. The method can include placing a jacket (see, e.g., jacket 293 in FIG. 2C) over the catheter liner, the actuation line assembly, and the braid. And the method can include reflowing the jacket to the catheter liner, the actuation line assembly, and the braid. In embodiments, the method can include placing a reinforcement member into the at least one keyway of the keyed mandrel.

1. A steerable catheter comprising:
a first actuation line;
an inner catheter having an inner catheter proximal end, an inner catheter distal end, an inner catheter outer surface defining a keyway that is longitudinally-extending, and an inner catheter inner surface defining an inner catheter lumen; and
an outer catheter having an outer catheter proximal end, an outer catheter distal end, an outer catheter outer surface, and an outer catheter inner surface defining an outer catheter lumen and a spline that is longitudinally-extending and that projects radially inward into the outer catheter lumen, the outer catheter lumen being operable to receive the inner catheter with the spline of the outer catheter operable to key with the keyway of the inner catheter, the spline defining a spline lumen operable to receive the first actuation line.

2. The steerable catheter of claim 1, further comprising an outer catheter actuation ring coupled to the outer catheter distal end, the first actuation line being coupled to the outer catheter actuation ring.

3. The steerable catheter of claim 1, further comprising a second actuation line, wherein the inner catheter defines an inner catheter actuation line lumen from the inner catheter proximal end to the inner catheter distal end and operable to receive the second actuation line therethrough, the second actuation line extending from the inner catheter proximal end to the inner catheter distal end and coupled thereto.

4. The steerable catheter of claim 3, further comprising an inner catheter actuation ring coupled to the inner catheter distal end, the second actuation line being coupled to the inner catheter actuation ring.

5. The steerable catheter of claim 1, wherein each of the spline and the keyway is parallel with a central axis of the steerable catheter.

6. The steerable catheter of claim 1, wherein the inner catheter includes at least one locking collar that defines the keyway of the inner catheter.

7. The steerable catheter of claim 1, wherein the inner catheter comprises a first elongated tubular member defining the inner catheter outer surface and the keyway is defined by the first elongated tubular member.

8. The steerable catheter of claim 1, wherein the outer catheter inner surface further defines a plurality of splines and the inner catheter outer surface further defines a plurality of respective keyways.

9. The steerable catheter of claim 1, wherein the outer catheter inner surface defines two splines that are located about 180 degrees apart on an outer catheter circumference and the inner catheter outer surface further defines two respective keyways located 180 degrees apart on an inner catheter circumference.

10. The steerable catheter of claim 1, wherein the outer catheter inner surface defines three splines that are located about 90 degrees apart on an outer catheter circumference and the inner catheter outer surface further defines three respective keyways located about 90 degrees apart on an inner catheter circumference.

11. The steerable catheter of claim 1, wherein the outer catheter inner surface defines three splines that are located about 120 degrees apart on an outer catheter circumference and the inner catheter outer surface further defines three respective keyways located about 120 degrees apart on an inner catheter circumference.

12. The steerable catheter of claim 1, wherein the steerable catheter comprises a deflectable region at or around the outer catheter distal end, and wherein the keyway extends along a length of the inner catheter toward the inner catheter distal end and terminates proximate the deflectable region of the outer catheter.

13. The steerable catheter of claim 1, wherein the inner catheter is slidably received within the outer catheter along a length of the keyway.

14. The steerable catheter of claim 1, further comprising a hemostatic seal between the outer catheter inner surface and the inner catheter outer surface.

15. A steerable catheter comprising:
a first actuation line having a proximal portion and a distal portion;
an inner catheter defining a first elongated tubular member having an inner catheter proximal end and an inner catheter distal end, and an inner catheter outer surface and an inner catheter inner surface defining an inner catheter lumen;
a keyed locking collar comprising a keyway and coupled to the inner catheter; and
an outer catheter having an outer catheter proximal end, an outer catheter distal end, an outer catheter outer surface, and an outer catheter inner surface defining an outer catheter lumen and a spline that is longitudinally-extending and that projects radially inward into the outer catheter lumen, the outer catheter lumen being operable to receive the inner catheter with the spline of the outer catheter operable to key with the keyway of the inner catheter, the spline defining a spline lumen operable to receive the first actuation line, the first actuation line extending along the outer catheter and the distal portion of the first actuation line being coupled proximate the distal end of the outer catheter.

16. The steerable catheter of claim 15, wherein the keyed locking collar is integrally formed with the first elongated tubular member of the inner catheter.

17. The steerable catheter of claim 15, wherein the keyed locking collar radially extends beyond an outer diameter of the inner catheter.

18. The steerable catheter of claim 15, wherein a length of the keyed locking collar is less than a length of the inner catheter.

19. The steerable catheter of claim 15, wherein the outer catheter inner surface defines two splines that are located about 180 degrees apart on an outer catheter circumference and the inner catheter outer surface further defines two respective keyways located about 180 degrees apart on an inner catheter circumference.

20. The steerable catheter of claim 19, wherein the outer catheter inner surface defines three splines that are located about 90 degrees apart on the outer catheter circumference and the inner catheter outer surface further defines three respective keyways located about 90 degrees apart on the inner catheter circumference.

21. A method of treating a patient, the method comprising:
obtaining the steerable catheter of claim 1;
advancing the outer catheter into a body lumen;
advancing the inner catheter into the outer catheter thereby engaging the spline with the keyway; and
actuating the first actuation line to thereby cause movement at the outer catheter distal end.

22. The method of claim 21, wherein the steerable catheter further comprises a handle at the outer catheter proximal end, the handle being operatively coupled to the first actuation line such that actuation of the handle causes actuation of the first actuation line, and wherein actuating the first actuation line comprises performing at least one actuation of the handle.
